(19) United States
(12) Patent Application Publication
(10) Pub. No.: US 2004/0224329 A1
(43) Pub. Date: Nov. 11, 2004

(54) Three-dimensional solid phase extraction surfaces

(76) Inventors: Douglas T. Gjerde, Saratoga, CA (US); Christopher T. Hanna, San Francisco, CA (US); Liem Nguyen, San Jose, CA (US); Leon S. Yengoyan, San Jose, CA (US)

Correspondence Address: PhyNexus, Inc., Attn: IP Dept., Suite A, 3670 Charter Park Dr., San Jose, CA, 95136, US

(21) Appl. No.: 10/754,775

(22) Filed: Jan. 8, 2004

Related U.S. Application Data
(63) Continuation-in-part of application No. 10/434,713, filed on May 8, 2003.
(60) Provisional application No. 60/523,518, filed on Nov. 18, 2003.

(51) Int. Cl. C12Q 1/68; C12M 1/34

(52) U.S. Cl. 435/6; 435/287.2

(57) ABSTRACT

The subject invention provides extraction capillaries, wherein a substantial portion of the channel is coated with a 3-dimensional solid phase extraction surface that binds an analyte. In some embodiments the extraction matrix comprises a polymer backbone with an extraction agent bound thereto. Analytes of particular relevance include biomolecules, such as proteins, polynucleotides, lipids and polysaccharides. The invention further provides devices comprising the extraction capillaries, reagents for use in conjunction with the capillaries and devices, and methods for the production and use of the capillaries and devices.

### CROSS-REFERENCE TO RELATED APPLICATIONS

This application claims priority to and benefit of U.S. patent application Ser. Nos. 10/434,713, filed May 8, 2003, Ser. No. 10/733,534, filed Dec. 10, 2003, and U.S. Provisional Application No. 60/523,518, filed Nov. 18, 2003, all of which are incorporated by reference herein in their entirety.

### FIELD OF THE INVENTION

This invention relates generally to capillary channels coated with three-dimensional solid phase extraction matrices, and the use of such capillary channels for the extraction of analytes from solution. Analytes of particular interest include biomolecules such as polypeptides and polynucleotides.

### BACKGROUND OF THE INVENTION

It is becoming increasingly important for the life scientist to be able to purify and concentrate biomolecule samples in a small volume. This is particularly true in the area of proteomics. Many of the technologies employed in the study of proteomics (e.g., mass spectroscopy, protein chips, X-ray diffraction and NMR) require small volumes of relatively concentrated samples of purified protein. These proteins are often present at low concentration in the starting sample, thus requiring that the sample processing technology be able to concentrate the protein of interest with minimal sample loss.

Solid phase extraction is one of the primary tools for preparing protein samples prior to this sort of analysis. A particularly powerful form of this technology, described in U.S. patent application Ser. No. 10/434,713 (filed May 8, 2003), employs solid-phase extraction capillaries to purify and enrich samples of proteins and other analytes. The subject invention provides solid-phase extraction capillaries having three-dimensional solid phases extraction matrices. These extraction capillaries find utility in the methods described in the Ser. No. 10/434,713 application, as well as in other application described herein. It has been found that these three-dimensional matrices provide powerful advantages relative to a corresponding two-dimensional extraction surface.

### SUMMARY OF THE INVENTION

In some embodiments, the subject invention provides an extraction capillary channel, wherein a substantial portion of the channel is coated with a 3-dimensional solid phase extraction surface that binds an analyte.

In some embodiments, the analyte binding capacity of the 3-dimensional solid phase extraction surface is greater than could be achieved by a corresponding 2-dimensional solid phase extraction surface.

In some preferred embodiments, the solid-phase extraction surface comprises a polymer, which can be attached to the surface of the capillary channel by one or more covalent bonds, one or more non-covalent interaction, or a combination of covalent and non-covalent interactions. An example of non-covalent interaction is an electrostatic interaction.

For example, the polymer can be attached to the capillary channel by electrostatic interaction to a second polymer, wherein the second polymer is attached to the capillary channel. Polymers of the invention can be cross-linked or non-cross-linked, can be in the form of a bead, e.g., a latex bead. Examples of polymers include polysaccharides, such as dextran.

In some embodiments the 3-D extraction surface is accessible to penetration by relatively large biomolecules, e.g., biomolecules of a mass of about 2000 Da.

In some embodiments, an extraction agent is attached to the solid-phase extraction surface. Examples of extraction agent include an immobilized metal, a protein, or an antibody, e.g., Ni-NTA, Protein A or Protein G. The extraction agent can be covalently attached to the polymer.

In some embodiments, the analyte is a biomolecule, such as a protein.

In some embodiments, the capillary channel is fused silica capillary tubing.

The invention further provides a method for preparing an extraction capillary channel having a 3-dimensional extraction surface, comprising the steps of: providing a capillary channel bearing a first attachment group; and attaching an extraction polymer to said capillary channel by an interaction between said first attachment group and a second attachment group on said extraction polymer, wherein said extraction polymer bears an affinity group having an affinity for an analyte.

In some embodiments, said extraction polymer is attached to said capillary channel by formation of a covalent bond between said first and second attachment groups, e.g., by formation of an amide bond, an isourea bond or thioether bond.

In some embodiments, said extraction polymer is attached to said capillary channel by an electrostatic interaction between said first and second attachment groups.

The invention further provides a method for molecular open tubular solid phase extraction, the method comprising the steps of: adsorbing analyte molecules in a sample solution to the extraction surface of a fused silica extraction capillary tubing of claim 1, the capillary tubing having a total capillary volume; and desorbing a substantial portion of the analyte molecules from the extraction surface with a desorbent liquid passed through the capillary channel.

In some embodiments, the analyte molecules is desorbed with a Tube Enrichment Factor of at least 1.

In some embodiments, the direction of passage of the desorption solution through the column reversed during the desorption step.

In some embodiments, a wash solution is passed through the capillary channel between steps (a) and (b).

In some embodiments, the wash solution is any liquid present in the capillary channel is substantially displaced from the capillary channel by a gas before step (b). Optionally, the direction of passage of the gas through the column is reversed during displacement of the liquid.

In some embodiments, the extraction surface has an affinity binding agent bound thereto, and the affinity binding agents is: a chelated metal having a binding affinity for a biomolecule analyte; a protein having a binding affinity for a protein analyte; an organic molecule or group having a binding affinity for a protein analyte; a sugar having a binding affinity for a protein analyte; a nucleic acid having a binding affinity for a protein analyte; a nucleic acid or a sequence of nucleic acids having a binding affinity for a nucleic acid analyte; or a small molecule binding agent having a binding affinity for a small molecule analyte.

In some embodiments, the analyte concentration is increased at least 100 times.

In some embodiments, the analyte molecules are desorbed with a Tube Enrichment Factor from within a range from 1 to 400.

### DESCRIPTION OF SPECIFIC EMBODIMENTS OF THE INVENTION

The subject invention provides, inter alia, capillary channels coated with three-dimensional solid phase extraction matrices, and methods and reagents for using such capillary channels for the extraction of analytes from solution. Analytes of particular interest include biomolecules, such as polypeptides and polynucleotides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a molecule” includes a plurality of such molecules and reference to “the detection method” includes reference to one or more detection methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In accordance with the present invention there may be employed conventional chemistry, biological and analytical techniques within the skill of the art. Such techniques are explained fully in the literature. See, e.g., Antibody Purification Handbook, Amersham Biosciences, Edition AB, 18-1037-46 (2002); Protein Purification Handbook, Amersham Biosciences, Edition AC, 18-1132-29 (2001); Affinity Chromatography Principles and Methods, Amersham Pharmacia Biotech, Edition AC, 18-1022-29 (2001); The Recombinant Protein Handbook, Amersham Pharmacia Biotech, Edition AB, 18-1142-75 (2002); and Protein Purification: Principles, High Resolution Methods, and Applications, Jan-Christen Janson (Editor), Lars G. Ryden (Editor), Wiley, John & Sons, Incorporated (1989); Chromatography, 5^{th} edition, PART A: FUNDAMENTALS AND TECHNIQUES, editor: E. Heftmann, Elsevier Science Publishing Company, New York, pp A25 (1992); ADVANCED CHROMATOGRAPHIC AND ELECTROMIGRATION METHODS IN BIOSCIENCES, editor: Z. Deyl, Elsevier Science BV, Amsterdam, The Netherlands, pp 528 (1998); CHROMATOGRAPHY TODAY, Colin F. Poole and Salwa K. Poole, and Elsevier Science Publishing Company, New York, pp 394 (1991); F. Dorwald ORGANIC SYNTHESIS ON SOLID PHASE, Wiley VCH Verlag Gmbh, Weinheim 2002.

As summarized above, the subject invention provides capillary channel devices useful for the purification and enrichment of analytes, particularly biomolecules, as well as methods and reagents for their use and production, and kits that include the same. In further describing the subject invention, the subject devices and reagents are described first in greater detail, followed by a review of representative methods in which they find use and a review of the kits that include the subject compounds.

The capillary channel can be single or bundled tubing, or it can be one or more channels in a block or chip. The channels can be straight. They can be non-linear shapes in the form of coils or other curved shapes which will promote agitated flow through the channels. The channels can be straight wall, undulating, knitted, circular, knotted, coiled, a combination of coiling and reverse coiling or filled with large bead to promote transport to the tube surface, or a monolithic structure. Coiled tubes can be cut to length for a specific application single sample use, eliminating cross-contamination.

The capillary channel may be composed of a number of different materials. These include fused silica, polypropylene, polymethylmethacrylate, polystyrene, nylon, (nickel) metal capillary tubing, and carbon nanotubes. Polymeric tubes are available as straight tubing or multihole tubing (Paradigm Optics, Inc., Pullman, Wash.). Nickel tubing is available from Valco Instrument, Inc., Houston, Tex. Formation of carbon nanotubes has been described in a number of publications including Kenichiro Koga, et al., Nature, 412:802 (2001).

In some embodiments the capillary channel is an element of a chip or disk for example of the type commercially available from vendors such as Tecan Systems, Inc. (San Jose, Calif.) and Gyros, Inc. (Monmouth Junction, N.J.). Extraction via a disk-based preconcentrator is described, for example, by Tomlinson et al., J. Chromatography A, 1996, 744:3-15.

In some preferred embodiments the capillary channel is a capillary tubing. Fused silica capillary tubing (i.e., fused silica capillaries), and especially capillaries comprising synthetic fused silica, have been found to be particularly suited for use as the extraction capillaries of the present invention. The fused silica provides numerous silanol groups which serve as useful attachment points for the extraction surface chemistries described herein. Fused silica capillaries that are suitable for the purposes of this invention include those produced by Polymicro Technologies, LLC of Phoenix, Ariz. and SGE Inc. of Ringwood, Australia.

While coiling of capillary tubing is not necessary, it can be advantageous in certain embodiments of the invention. Coiling results in a more tortuous flow path, which can improve the efficiency of the extraction process. Another benefit of coiling is that it allows for the production of a relatively compact extraction device that would not otherwise be feasible, due to the length of the extraction capillary tubing.

Typically capillary tubing is used having a total outside diameter in the range of 90 to 3500, 90 to 1500, 90 to 850, 150 to 850 or 238 to 435 microns. Capillary channel internal diameters are typically in the range of about 2 to 3000 microns, about 2 to 1000 microns, about 10 to 700 microns, about 25 to 400 microns, or about 100 to 200 microns. In preferred embodiments of the invention the outer surface of the capillary tubing is coated with a flexible coating material, typically a polymer or resin. Preferred coating materials include polyimide, silicone, polyacrylate, aluminum or fluoropolymer, especially semiconductor grade polyimide. As used herein the term “overall diameter” refers to the total diameter of a capillary, including coating if any. The “outer diameter” refers to outer diameter of capillary minus any coating, e.g., the outer diameter of a fused silica capillary tubing.

The length of the capillary channel can vary greatly depending upon the desired capacity, contemplated sample size and desired enrichment. Because fused silica tubing can be coiled, relatively compact extraction devices can be constructed that include 1 meter or more of capillary tubing. Alternatively, in some embodiments very short lengths of capillary can be employed, down to 1 mm in length or even shorter.

When working with fused silica capillary tubing, particularly where the tubing is coiled, it is important to exert all possible care to coil avoid the introduction of nicks or other breakage that can lead to breakdown of extraction function. For instance, it is usually better not to introduce any twisting of the tubing during the coiling process, as this twisting will itself introduce stress into the tubing beyond that introduced by the coiling. Other precautions that will reduce breakage include minimizing nicks on inner and outer surface of capillary, the use of a thicker coating, preferably a polyimide coating, and minimizing exposure of the capillary surface to base.

The capillary channel can be a single tube or be formed as a block of multiple tubes or a multichannel block (multicapillary format).

The subject invention provides extraction capillaries having channel surfaces coated with a three-dimensional solid phase extraction surface that binds an analyte of interest. For many applications of the invention it is desirable that the surface bind tightly and specifically to a biomolecule (or class of biomolecules) of interest, especially relatively large biological macromolecules (e.g., polynucleotides, polypeptides and polysaccharides having a MW of greater than about 1000 Da, including, for example, in the range of 1000 to 10,000,000 Da or more, or more typically in the range of 5000 to 500,000 Da). For use in conjunction with biological samples it is desirable that the three-dimensional solid phase extraction surface forms a biocompatible porous surface. The porosity of the surface allows for the penetration of biomolecules such as proteins into the surface, and interaction of the biomolecules with affinity groups present in the surface. In some preferred embodiments the extraction surface is based upon a fluidic, hydrogel-type environment. Such an environment is particularly suited for the extraction and purification of proteins, since it mimics the properties of bulk solution and can help stabilize the protein in its active form, i.e, the conditions are non-denaturing. Depending upon the particular properties of the analyte, non-limiting examples of suitable surface materials for providing the 3-D structure include porous gold, sol gel materials, polymer brushes and dextran surfaces.

The three-dimensional surface layer typically has a thickness of from a few angstroms to thousands of angstroms. In some embodiments the surface is between 5 to 10,000 angstroms thick, e.g., 5 to 1000 angstroms. The thickness of the surface can be adjusted as desired based on factors including the dimensions of the capillary channel, the nature of the analyte or analytes of interest, the nature of an affinity group or extraction reagent present in the surface, the desired binding capacity, etc.

In some embodiments of the invention the 3-D solid phase extraction surface is a hydrogel formed from a polymer, e.g., a polysaccharide or a swellable organic polymer. The polymer should be compatible with the analyte of interest and with a minimal tendency towards nonspecific interactions. Examples of suitable polysaccharides include agarose, sepharose, dextran, carrageenan, alginic acid, starch, cellulose, or derivatives of these such as, e.g., carboxymethyl derivatives. In particular, polysaccharides of the dextran type which are non-crystalline in character, in contrast to e.g., cellulose, are very suitable for use in the subject invention. Examples of water-swellable organic polymer would include polyvinyl alcohol, polyacrylic acid, acrylate, polyacrylamide, polyethylene glycol, functionalized styrenes, such as amino styrene, and polyamino acids. Exemplary polyamino acids include both poly-D-amino acids and poly-L-amino acids, such as polylysine, polyglutamic acid, polyaspartic acid, co-polymers of lysine and glutamic or aspartic acid, co-polymers of lysine with alanine, tyrosine, phenylalanine, serine, tryptophan, and/or proline.

Desirable functional attributes of the 3-D surface would include that it should have minimal tendency to interact non-specifically with biomolecules, it should be chemically resistant to the media employed, it should be compatible with proteins and other biomolecules and should not interact with any molecules other than those desired. Furthermore, it should be capable of providing for covalent binding of such a large number of affinity groups as is required for a general applicability of this technique to a variety of analytical problems.

For a number of reasons, dextran, dextran-derivatives and dextran-like materials are particularly suited for use as the backbone molecules in the subject 3-D extraction surfaces. The resulting hydrogel layer is highly flexible, largely non-cross linked and typically extends 100-200 nm from coupling surface under physiological buffer conditions. Dextran can be derivatized, e.g., via carboxymethylation or vinylsulfonation, to incorporate additional reactive handles for activation and covalent attachment of affinity groups. Non-limiting examples of coupling chemistries that can be used with these and related backbone molecules include thiol, amine, aldehyde and streptavidin. See, e.g., F. Dorwald ORGANIC SYNTHESIS ON SOLID PHASE, Wiley VCH Verlag Gmbh, Weinheim 2002, Anal. Biochem. (1991) 198 268-277 and Chem Commun. (1990) 1526-28). These chemistries are generally quite robust. One potential disadvantage of dextran is it's negative charge, which can result in undesired interactions with charged proteins depending upon the pH and ionic strength of the environment. This factor can typically be dealt by adjusting parameters to minimize any unwanted non-specific interactions.

The polymer used to form the extraction surface can be cross-linked, e.g., cross-linked dextran. The degree of cross-linking can be varied to adjust the porosity and hence accessibility of the extraction surface, particularly to larger molecules such as biological macromolecules. In many instances, however, it will be desirable to employ minimal or no cross-linking, e.g., low cross-linked dextran, to provide improved accessibility into the surface and improved transport properties. This can be important in procedures wherein a small volume of elution solvent are used to achieve a low volume, highly concentrated sample of analyte. While it can be difficult to prepare a polymer-based 3-D extraction surface without the occurrence of some incidental cross-linking, minimal or low crosslinking can be achieved using methods exemplified in this written description. This differs from conventional columns that use more highly cross-linked polymers. In general, the lower the extent of cross-linking the more accessible the extraction surface is to analyte penetration.

In preparing 3-D extraction surfaces on capillary surfaces there is typically greater latitude with regard to the degree of cross-linking permitted relative to the beads used in conventional chromatography. Generally polymer-based beads require a certain degree of cross-linking to maintain their structure, particularly in the presence of the pressure that develops during the chromatographic process. For example, conventional Sepharose chromatography beads require a certain degree of cross-linking in order to prevent bead distortion and collapse due to the flow pressure. The 3-D extraction surfaces of this invention, being present on the surface of an open channel and thus not subject to the same pressures as beads in a packed column, are generally not restricted to any minimum degree of crosslinking. Thus, extractions surface backbones that have no or low degree of cross-linking can be used, resulting in greater accessibility of the extraction surface to analyte, particularly high MW biomolecules. Thus, extraction surfaces comprising a polymer backbone that is, for example, less than 0.1% crosslinked, about 0.1 to 0.5% crosslinked, about 0.5 to 1% crosslinked, about 1 to 2% crosslinked, about 2 to 3% crosslinked, about 3-5% crosslinked, about 5-7% crosslinked, about 7-10% crosslinked, or even greater than 10% crosslinked can be used. The acceptable degree of crosslinking varies depending upon the nature of the polymer backbone (e.g., swellability of the polymer) and the nature of the analyte (e.g., size and structure of a biomolecule, the molecules hydration volume). Because crosslinking is not required, a variety of backbone chemistries may be employed that would not be appropriate for use in a conventional chromatography bead.

In some embodiments of the invention, the interior of the 3-D extraction surface is accessible to analyte, such that analyte molecules are able to penetrate and adsorb to the surface in 3-dimensions. In particular, some embodiments are accessible to relatively large biological macromolecules, e.g., polynucleotides, polypeptides and polysaccharides having a MW of greater than about 1000 Da, including, for example, in the range of 1000 to 10,000,000 Da or more, or more typically in the range of 5000 to 500,000 Da (e.g., biomolecules of 1000 Da, 2000 Da, 5000 Da, 10,000 Da, 50,0000 Da, 100,000 Da, 500,000 Da, 1,000,000 Da, etc.). This can be particularly useful for the extraction of biomolecule complexes, e.g., complexes comprising two or more proteins bound to one another by covalent or non-covalent interactions, a protein bound to a polynucleotide, etc. It is known that many clinically relevant biomolecules function as part of such complexes, which can in some cases be quite large. Thus, one advantage of the subject invention is that it facilitates the study of such complexes.

With regard to the extraction of biomolecule complexes, in some embodiments the invention provides methods for purifying and characterizing such complexes. For example, a complex of interest can be adsorbed to the extraction surface, and then components of the complex selectively desorbed and collected, and optionally subjected to further characterization, e.g, by MS, NMR or SPR. The non-denaturing conditions of the 3-D extraction surfaces lend themselves particularly to this type of analysis, since often times these biomolecule complexes are quite fragile.

Properties of a 3-D extraction surface of the invention, including thickness and porosity, can be modified by varying the MW (or MW range) of the polymer backbone. Polymers in the MW range from about 500 to several million can be used, preferably at least 1000, for example in the range of 10,000 to 500,000. In some cases an increase in MW can result in improved performance, e.g., higher capacity. For example, dextran is available in a variety of MW ranges, allowing for modification of physical characteristics of the resulting hydrogel. Properties of the hydrogel can also be modified by variation of functional groups, extent and nature of cross-linking, etc.

In order to function as a solid phase extraction medium, the 3-D surface should have an affinity for an analyte of interest. In some embodiments the affinity is strong and selective, resulting in a substantially single equilibrium absorption of the analyte under extraction conditions. The affinity can be inherent to the surface itself, or more typically the result of attachment of an affinity group to the surface backbone. As used herein, the term “affinity group” refers to a chemical entity having an affinity for an analyte of interest, e.g, a biomolecule. Types of affinity groups include ion exchangers, which can be strong or weak (e.g., acids, bases, quaternary amines). Other types of affinity groups include polar or non-polar groups, e.g., hydrophobic or reverse phase groups. The affinity group is often an extraction agent able to bind specifically to a biomolecule analyte of interest, e.g., a specific polypeptide or class of polypeptides, a specific polynucleotide or class of polynucleotides, a polysaccharide, a lipid, a metabolite or other small molecule. While the interactions between an analyte and an affinity group can be specific or relatively non-specific, e.g., attraction based on electrostatic or hydrophobic interaction, an extraction agent is characterized by more specific interaction.

Extraction agents include various ligands such as metal chelators (and the corresponding immobilized metal ions), antibodies, proteins, polynucleotides, etc. A detailed description of a wide variety of solid phase affinity groups and extraction agents is provided in U.S. patent application Ser. No. 10/434,713, along with guidance for making and using extraction columns employing the same. In general, these affinity groups can be used in a like manner with the 3-D extraction matrices of the subject invention. Non-limiting examples of some particularly useful extraction agents include metal chelators used in immobilized metal affinity chromatography (e.g, metal-IDA, metal-NTA, metal-CMA), Protein A, Protein G, avidin or streptavidin (monomeric or multimeric), calmodulin, glutathione, maltose and antibodies having an affinity for an epitope tag.

In some embodiments the extraction surface contains a functional group or groups suitable for use in the attachment of an affinity group, e.g., an extraction agent. Representative examples of such groups include hydroxyl, carboxyl, amino, aldehyde, ketone, carbonyl, activated ester, epoxy and vinyl groups. These functional groups are also useful for attachment of the extraction surface to the surface of the capillary channel, or can be used for cross-linking the matrix itself. In some embodiments, two or more different functional groups are used, e.g., one for attachment of extraction agent or extraction agents, another for attachment of matrix to channel surface. In another non-limiting example, the same functional group can be used for attachment of the matrix to extraction agents and channel surface.

If a desired functional group is not inherently present in the 3-dimensional matrix backbone it can be introduced synthetically, e.g., the carboxymethylation of dextran to introduce carboxyl groups, as described in the appended examples. Methods to attach chemical groups to polymers are described in the following organic synthesis texts, and these texts are hereby incorporated by reference herein in their entireties, Jerry March, ADVANCED ORGANIC CHEMISTRY, 3^{rd} ed., Wiley Interscience: New York (1985); Herbert House, MODERN SYNTHETIC REACTIONS, 2^{nd} ed., Benjamin/Cummings Publishing Co., California (1972); Jansen and Ryden, editors, PROTEIN PURIFICATION: PRINCIPLES, HIGH RESOLUTION METHODS, AND APPLICATIONS VCH Publishers Inc. (1989); and James Fritz, et al., ION CHROMATOGRAPHY, 3rd, ed., Wiley-VCH, New York (2002).

In some embodiments of the invention it is desirable to prepare an extraction matrix including a functional group in an activated form, e.g., an activated carboxyl. This activation facilitates the coupling of an extraction agent of interest to the matrix, e.g,. via formation of an amide bond. For example, an activated carboxyl group can take any of a number of forms, including but not limited to activated reactive esters, hydrazides, thiols or reactive disulfide-containing derivatives. A reactive ester can be prepared in any of a number of ways known to one of skill in the art, including by reaction with a carbodiimide. In one embodiment the activated functional group is a 2-aminoethanethiol derivative. In yet another embodiment the activated functional group is a vinyl sulfone.

In one embodiment, a hydrazide function can be created in dextran matrix for binding ligands containing aldehyde groups, for example antibodies in which the carbohydrate chain has been oxidized so that it then contains an aldehyde function. The dextran matrix is initially modified with, e.g., carboxymethyl groups, which are subsequently reacted to form hydrazide groups.

According to another embodiment, carboxyl groups in carboxymethyl-modified dextran are modified so as to give reactive ester functions, e.g., by treatment with an aqueous solution of N-hydroxysuccinimide and N-(3-dimethylaminopropyl)-N′-ethylcarbodiimide hydrochloride. Ligands containing amine groups such as, for example, proteins and peptides may then be coupled to the dextran matrix by covalent bonds.

According to an alternative procedure, the aforesaid reactive ester is utilized for reaction with a disulfide-containing compound such as for instance 2-(2-pyridinyldithio) ethanamine; in this manner a matrix is obtained which contains disulfide groups, and these can be employed for coupling thiol-containing ligands such as for example reduced F(ab) fragments of immunoglobulins. After cleavage of the disulfide bonds, for instance by reduction or thiol-disulfide exchange, the thiol modified surface formed can be used for coupling of a disulfide-containing ligand such as, for instance, N-succinimidyl 3-(2-pyridinyldithio) propionate (SPDP) modified proteins.

The invention provides methods for preparing extraction capillary channels having 3-dimensional extraction surfaces. In one approach, the extraction surface is prepared by attaching an extraction polymer (e.g., a polymer bearing an affinity group as described herein) to a capillary channel. The attachment is accomplished by means of an interaction between complementary attachment groups on the polymer and channel. The term “complementary” refers to the ability of the attachment groups to interact with one another in such a way as to result in attachment of the polymer to the channel. Examples of such interactions include electrostatic attraction (e.g., where the attachment groups are oppositely charged ions) and hydrophobic interactions (e.g., where the attachment groups are non-polar groups that are attracted to one another in a polar environment. The interaction can be one that results in the formation of a covalent bond, e.g., the complementary attachment groups are functional groups capable of forming covalent bonds, e.g., a carboxyl group and an amide group are complementary functional groups capable of reacting to form an amide bond, vinyl and thiol are complementary functional groups capable of reacting to form a thioether bond. Other examples of complementary groups are cyanogen bromide and the amine group, which can react to form an isourea bond (Porath et al. (1973) J. Chromatograph. 86:53; and Kohn and Wilchek (1984) Appl Biochem. Biotechnol. 9:285-304), and maleimide and thiol, which can react to form a thioether bond (Wang et al. (2003) Bioorganic and Medicinal Chemistry 11: 159-6; Toyokuni et al. (2003) Bioconjugate Chem. 14:1253-59; Frisch et al. (1996) Bioconjugate Chem. 7:180-86). The maleimide reaction is particularly useful in certain embodiments of the invention for attaching a group to a polydextran matrix with minimal crosslinking of the matrix. The maleimide group is relatively specific for the thiol group, and not prone to unintended reaction with the dextran matrix. Use of the maleimide group as a linker is exemplified further in the examples, where preparation of a polymaleimide dextran is described. This polymaleimide dextran can be a particularly low-crosslinked matrix, which can be more easily penetrated by some larger molecules, as described elsewhere herein.

The attachment of an extraction polymer to a capillary channel can be direct, but more typically is accomplished by one or more linker molecule that serves as intermediaries bridging the polymer and the surface of the extraction channel. Attachments between polymer and linker, linker and channel surface, and/or linker to linker can be covalent or non-covalent. The linker molecule can itself be a polymer, or not. For example, the linker molecule can be a polymer that interacts with the capillary channel and with the extraction polymer, bridging the two. When the capillary channel is silica, for example, surface of the channel is normally covered with silanol groups, resulting in a net negative charge to the surface. A bridge molecule having a positive charge (e.g., a polymer, such as a strong base anion exchanger) can be used to coat the surface, attached thereto by electrostatic attraction. An extraction polymer having a negative charge (e.g., a cation exchanger) can then be attached to the surface through the bridging molecule, in this case by electrostatic attraction to the positively charged bridging polymer. Note that this embodiment involves the successive stacking of layers of polymer having opposite charge on the capillary surface. The number of layers can be one, two or more. For example, successive layers of oppositely charged polymers can be coated on the surface of the capillary channel, with the last applied (or top) layer constituting the extraction surface. In some preferred embodiments the extraction polymer and/or bridge polymers are beads. These beads can be held together by cross-linking (or not). Latex beads are used for this purpose in some of the Examples.

When employing a silica capillary, it is often convenient to covalently couple the matrix to the capillary through free silanol groups on the channel surface. This is typically accomplished through a linking molecule bridging the silanol group and matrix backbone, e.g., polymer. For example, reactive thiol or amino groups can be attached via reaction with a thiosilane or aminosilane, respectively. A carboxyl group can be introduced on the capillary surface by reaction of amino-functionalized capillary with an anhydride, e.g., succinic anhydride.

In another embodiment, a three-dimensional matrix can be attached to a capillary surface through a self-assembled monolayer. This is particularly useful where the capillary is metal, e.g., gold. The attachment of a matrix to a metal surface through a self-assembled monolayer has been described elsewhere, see, for example U.S. Pat. Nos. 5,242,828; 6,472,148; 6,197,515 and 5,620,850.

In an alternative embodiment, a 3-D polymer matrix can be attached through the SMIL (successive multiple ionic-polymer) approach as described by Katayama et al. (1998) Analytical Sciences 14:407-409.

An advantage of the 3-D extraction surfaces of the subject invention is their high surface area relative to a corresponding 2-D extraction surface (i.e., monolayer), which allows for improved analyte binding capacity. That is, the 3-D matrix allows for denser placement of affinity groups (e.g., extraction agents) per surface area of the capillary channel (or length of capillary channel), and/or for denser binding of analyte. For an example of a 2-D extraction surface, or monolayer, see Cai et al. (1993) J. of Liquid Chromatography 16(9&10) 2007-2024, who report fused-silica capillaries having surface-bound iminodiacetic acid metal chelating functions. Note that the support coated capillaries prepared by Cai et al. using a colloidal silica solution do not exhibit the increased capacity of the preferred 3-D extraction matrices of the subject invention, since the silica coating is not swellable (i.e., does not take up water or solvent like polysaccharide polymer such as dextran) and cannot be substantially penetrated by high MW biological macromolecules. Note that the concept of a 2-D monolayer does not necessarily imply a flat surface, since a monolayer surface can be rough or have contours that in some cases can provide some increase in capacity. A 3-D matrix, on the other hand, is penetrable. The capacity of a 2-D binding surface will depend on the diameter of the analyte molecule and the ability of the molecules to “close pack” together. “Close pack” refers to the situation where sides of the analyte molecules are touching or nearly touching each other on a 2-D surface. One way of considering the subject invention is that a 3-D binding phase allows for packing of analyte molecules on a 3^{rd} dimension. This packing can be a close pack or approach a close pack in three dimensions. The magnitude of the increased capacity compared to a monolayer follows from the ability of the binding phase to capture analyte molecules in the third dimension.

The three-dimensional nature of the matrix is particularly advantageous in that it allows for much higher binding capacity of large biomolecules such as proteins. To illustrate, consider the binding of a globular protein analyte to a 2-dimensional, monolayer extraction surface. The binding of the globular protein creates a “footprint” on the surface where no other protein is able to bind. In the case of a corresponding 3-D surface, the protein can bind in the matrix at varying distances from the channel surface, allowing for a staggering of the proteins and the capacity to bind many more proteins than would be possible on a 2-D surface in a capillary channel of comparable dimensions. Representative data demonstrating the substantial improvement in protein binding capacity of a 3-D extraction matrix relative to a corresponding 2-D extraction matrix is provided in the Examples. As used in this sense, the term “corresponding” refers to matrices sharing the same affinity group (e.g., extraction agent), the difference between the corresponding matrices being that one is 2-D while the other is 3-D.

Another advantage of the 3-D extraction surface is that it can provide a more gentle and hospitable environment for delicate biomolecules (e.g., large proteins and protein complexes) compared to a 2-D surface. The 3-D matrix allows for the creation of an environment that more closely mimic the properties of bulk solution. This biomolecule-friendly environment can promote protein stability and the retention of native biological activity.

In some embodiments of the invention, particularly those involving the extraction of proteins, it can be desirable to perform the extraction at a temperature and under conditions that stabilize the functional protein, e.g., non-denaturing conditions. For example, most proteins are more stable at moderate to low temperatures, e.g., at a temperature of less than 60° C., preferably in a range of around 0 to 40° C., 0 to 25° C., 0 to 10° C., 0 to 4° C., 2 to 40° C., 2 to 25° C., 2 to 10° C., 2 to 4° C., 4 to 20° C., or 4 to 10° C. Functional proteins can also be stabilized by control of pH using a buffer adjusted to a pH range suitable for the analyte of interest (if known). In many cases a neutral pH (pH 7) or pH around neutral (pH 4 to pH 10) will be best, but this can vary depending upon the nature of the analyte, e.g, the pI of a protein.

In some preferred embodiments of the invention the extraction capillary is a component of one of the extraction devices described in U.S. patent application Ser. No. 10/434,713. Alternatively, the extraction capillary can be used as an open tubular chromatography column by adapting conventional chromatographic methodologies to the capillary.

An advantage of performing extractions in a capillary channel as opposed to a conventional packed column is that solvent can flow through the column at a much higher linear velocity. For example, in a typical Protein A affinity packed column of dimensions 0.7×2.5 cm (1 mL) about half the column volume is taken up by resin. Therefore for a (typical) flow rate of 1.0 mL/min the linear velocity of the fluid flow is 5 cm/min. For a 200 μm i.d.×1 m length capillary, the column volume is 33 μL. When sample is processed, about 1000 μL of sample is moved through the capillary in 10 min. This means the flow 0.1 mL/min, corresponding to a linear velocity of 300 cm/min.

Generally, the effective capacity of a column will decrease as the flow rate is increased. See, for example, Samuelson, O., “Ion Exchange Separations in Analytical Chemistry” (John Wiley and Sons, 1963) page 97 et seq.; and Kunin, R., “Ion Exchange Resins, 2^{nd} Ed.” (John Wiley and Sons, 1958) page 339 et seq. This is especially true for gel resins. As the flow is increased the transport time for the analyte to penetrate the bead and interact with the functional group becomes too long and the effectiveness of these internal groups decreases. Thus, it is surprising to find that the 3-D extraction matrices of the invention function effectively as extraction phases at these high linear velocities.

The capillaries can also be used in multiplexed or parallel operations, especially when used in conjunction with automated and/or computer-controlled apparatuses, e.g., robotic instruments.

In some preferred embodiments of the invention the extraction capillary, or a device comprising same, is used in a separation method or procedure as described in U.S. patent application Ser. No. 10/434,713. Particularly preferred are applications that result in a tube enrichment factor (TEF) of greater than one, since these have the potential to provide particularly high analyte concentration with minimal sample loss. The term “solid phase extraction tube enrichment factor” or “TEF” is defined as the ratio of the volume of a channel, to the volume of the liquid segment containing the desorbed analyte. The term “liquid segment” is defined herein as a block of liquid in a channel, bounded at each end by a block of liquid or gas. Alternatively, the subject extraction capillaries can be adapted for use as open tubular chromatography column for use in conventional chromatographic applications. TEFs of one are higher can be achieved using the methods of the invention, for example TEFs in the range of 1 to 10, 1 to 100, 1 to 400, 1 to 1000 or even higher in some cases.

The term “solid phase extraction enrichment factor” is defined as the ratio of the volume of a sample to the volume of liquid segment containing the desorbed analyte. Thus, the enrichment factor takes advantage not only of the TEF, but also the ability to run a large volume of sample through the capillary during the loading step, and optionally to run the sample back and forth through the capillary multiple times during the sample adsorption step. This is particularly advantageous where the analyte of interest is present at a low level in the sample solution.

The extraction capillaries and devices of the inventions can be used in a variety of methods for extraction, typically resulting in purification and/or concentration of the analyte. These methods can be performed by loading the sample into the capillary channel from either end, washing the capillary channel from either end, and desorbing with a segment of solvent from either end, where the segment containing desorbed protein(s) or biomolecules(s) is directed to or deposited on a target. The target can be a spot on a protein chip device.

In some embodiments, the method used involves attaching one end of an extraction capillary to a pump capable of pumping liquid and/or gas, and introducing sample solution containing an analyte of interest into the second end of the capillary by contacting the second end with a sample solution and activating the pump. The volume of sample solution can be much larger than that of the capillary, or in some cases smaller. The ability to pass a larger volume of sample solution through the capillary can be useful in the case where the analyte is present at low concentration. By running the sample through the capillary at an appropriate flow rate adsorption of the analyte to the extraction surface can be achieved. In some cases it may be desirable to pass the sample solution back and forth through the capillary, allowing for increased exposure to extraction surface and potentially greater extent of binding. The flow rate can also be reduced to allow more time for interaction between the analyte and matrix. An advantage of the invention is that generally higher flow rates can be used than with a corresponding conventional packed bed extraction matrix.

After passing through the capillary (or at least some portion of the capillary) one or more times, the sample solution is substantially displaced from the capillary. Displacement is typically achieved by introducing a gas into the capillary, e.g., a pump can be used to blow or suck air through the capillary, or centrifugation or a vacuum pump could be used to achieve a like result. Alternatively, the sample can be displaced by a wash solution. The wash solution is useful for removing unwanted contaminants prior to the desorption step. Gas can be run through the column prior to and/or subsequent to the wash step, to remove any residual liquid from the capillary. The passage of gas through the column allows for improved purification and concentration of the sample. Gas can be run through the capillary in one direction, or the direction of gas flow can be reversed one or more times during the process. In some embodiments the gas is nitrogen gas, run through the capillary at a sufficient pressure and for as sufficient time to substantially remove any liquid from the capillary, e.g., at 50-60 psi for 30-60 seconds. Likewise, any liquid solution passed through the capillary can run through the capillary in either or both directions, and flow can be reversed one or more times. In some embodiments sample, wash and/or desorption solutions enter and exit the channel through the same opening, as opposed to flowing in one end of the capillary and out the other as in other forms of chromatography.

After the adsorption and optional washing steps, the analyte is desorbed by introduction of desorption solution into the capillary, preferably flowing the desorption solution through the capillary one or more times throughout the entire length of the capillary to which analyte is adsorbed. A small plug of desorption solution having a volume equal to or less than that of the capillary can be used to achieve a Tube Enrichment Factor of one or greater. The desorption solution can enter and exit the capillary through same opening. In some cases analyte recovery can be improved by running the plug of desorption solution back and forth through the capillary one or more times. It follows that it is desirable to use a pump that is capable of precisely aspirating a small slug of desorption solution (of desired quantity) and accurately manipulate the slug in the capillary so as to achieve maximal elution of analyte in a minimal volume. As noted elsewhere herein, high recovery of concentrated sample is particularly desirable when the analyte is to be subjected to further analysis, e.g., by MS, X-ray crystallography, NMR, SPR, etc.

The invention also provides a device comprising an extraction capillary channel having a first end and a second end, the first end being connected to a pump for pumping liquid and gas. The pump can be, e.g., a syringe, pressurized container, centrifugal pump, electrokinetic pump, or an induction based fluidics pump. For some applications, the second end can be connected to an interface for a protein chip sample applicator or a mass spectrometer.

The subject invention also includes kits including one or more of the subject extraction capillaries, and optionally including ancillary reagents and devices for use in conjunction with said capillaries, such as wash, loading and/or elution solutions, pumps, etc.

Often times it will be practical to use the capillaries of the invention as disposable products, since they can be provided at a relatively low cost using the procedures described herein. An advantage of disposable capillaries is that it avoids the problems of contamination and carry-over from one sample to the next. This is a distinct advantage over many alternative methodologies, chromatographic and otherwise, which do not lend themselves to use as disposable elements. For example, conventional HPLC columns are generally too expensive not to be reused, leading to tedious and time consuming cleaning and regeneration steps between samples and the potential for carry-over of contaminants from a previous experiment.

Having now generally described the invention, the same will be more readily understood through reference to the following examples, which are provided by way of illustration, and are not intended to be limiting of the present invention, unless so specified.

### EXAMPLES

The following preparations and examples are given to enable those skilled in the art to more clearly understand and practice the present invention. They should not be construed as limiting the scope of the invention, but merely as being illustrative and representative thereof.

### Example 1

Hydroxide Etch Conditioning of Fused Silica Capillary Tubing

Fused silica capillaries (204 μm ID, 362 μm OD; 50 meters×2; obtained from Polymicro Inc. (Phoenix, Ariz., lot #PBW04A) were etched by flowing 100 mM NaOH through the capillary at a flow rate of 0.05 mL/min for 50 minutes. The capillaries were then washed with water (6.0 mL), 0.1N HCl (2 mL), water (10 mL) and acetonitrile (6 mL), after which they were dried with nitrogen gas.

### Example 2

Synthesis of Amino-Functionalized Capillary

A 10 meter section of the etched capillary described in Example 1 was filled with a solution of (MeO)₃Si(CH₂)₃NH₂ (400 μL) in toluene (1200 μL). The capillary was placed in a 120° C. oil bath and the reaction continued for 16 hrs with the flow of the silanization solution through the capillary adjusted to 0.8 μL/min. The capillary was then washed with toluene (1000 μL), acetonitrile (2000 μL), and dried with nitrogen at room temperature.

### Example 3

Synthesis of Carboxylic Acid-Functionalized Capillary

A four meter length of the amino-functionalized capillary described in Example 2 was filled with a solution containing succinic anhydride (125 mg; 1.25 mmol), DMAP (20 mg), pyridine (25 μL) in DMF (400 μL) and acetonitrile (900 μL). The capillary was placed in a 65° C. oven and the reaction continued for 15 hrs with the flow of the succinic anhydride solution adjusted to 0.6 μL/min. The capillary was then washed with acetonitrile (2000 μL).

### Example 4

Synthesis of “Nitrilotriacetic Acid” (NTA)

N,N-Bis-(carboxymethyl) lysine (commonly referred to as “Nitrilotriacetic acid,” or “NTA”) was synthesized as follows based the procedure reported by Hochuli et al. (Journal of Chromatography, 411:177-184 (1987)).

A solution of H-Lys(Z)-OH (42 g; 150 mmol) in 2 M NaOH (225 mL) was added dropwise to a solution of bromoacetic acid (42 g; 300 mmol; 2 eq) in 2 M NaOH (150 mL) at ˜0 to 10° C. White precipitate formed as the solution of H-Lys(Z)-OH added. The reaction continued at room temperature (RT) overnight, after which the temperature was increased to 60° C. and the reaction continued for another 2 hrs. Hydrochloric acid (1M, 450 mL) was added and the mixture was placed in a refrigerator for a couple of hours. The solid product (Z-protected NTA) was filtered off and recrystalized by re-dissolving the solid in 1 M NaOH, then neutralized with the same amount of 1 M HCl. The Z-protected NTA was collected by filtration and dried.

Z-protected NTA was dissolved in 1 M NaOH (130 mL) and 5% Pd/C (˜450 mg) was added. The reaction mixture was evacuated and saturated with H₂ before being stirred at RT under H₂ balloon overnight. The reaction mixture was filtered through a celite bed to remove the Pd/C. The filtrate, containing NTA was collected and water (80 mL) was used to wash the filtering bed. Hydrochloric acid (1M, 450 mL) was added to bring the pH down to 7.5-8.0. The collected NTA solution was diluted with water to give a final concentration of about 200 mM.

### Example 5

Synthesis of an Extraction Capillary Coated with an NTA Monolayer

A four meter length of the carboxyl-functionalized capillary described in Example 3 was activated by filling the capillary with a solution of N-hydroxysuccinimide (115 mg, 1.0 mmol), and EDAC (191.7 mg, 1.0 mmol) in acetonitrile (1500 μL). The reaction continued for 3 hrs at RT with the flow of the above solution through the capillary adjusted to 5 μL/min. (The reaction can also be carried out for about 14 hrs with the flow of the reagents solution adjusted to 0.6 μL/min.)

The activated capillary was washed with acetonitrile (1000 μL), then treated with a solution of NTA (described in Example 4) in water (200 mM, pH˜8, 1.0 mL). The reaction continued for 14 hrs at RT with the flow rate adjusted to about 1 μL/min. The capillary was further reacted with 0.5% ethanolamine in water for 2 min before it was washed with water (4 mL).

### Example 6

Charging a NTA Extraction Capillary with Ni²⁺

An extraction capillary coated with NTA monolayer as described in Example 5 was washed by flowing 500 μL of 100 mM NaHCO₃ through the capillary at a fast flow rate. The washed capillary was then charged with 10 mM NiSO₄ for 20 min (flow rate ˜20 μL/min). The charged capillary was then washed with water (1 mL at a fast flow rate), followed by 10 mM NaCl (500 μL; 50 μL/min), and then a final water wash (6 mL; 100 μL/min). Toward the end of the final water wash the effluent was spot checked with PAR reagent (pyridine azoresorcinol) for the presence of any Ni²⁺ (see Example 18).

The capillary was then cut into 1 meter lengths each for use in extraction procedures.

Capillaries that have been used in extractions can be recharged using the same procedure. Prior to recharging a capillary it should be washed with 50 mM Na₂EDTA (500 μL; fast with about 1 min of incubation).

### Example 7

Synthesis of Polycarboxymethyl Dextran

Dextran (ICN Cat# 101507; MW. 15000-20000; 3 g) was dissolved in 60 mL of water (with the help of a heat gun) and bromoacetic acid (9.3 g) was added followed by Ag₂O (8.6 g). The reaction was allowed to continue at RT for 24 hrs. The Ag₂O was not completely dissolved, so the reaction looked like it contained charcoal. This charcoal color eventually turned to milky-brown. The reaction stopped and solid material was filtered over celite. The filtrate was dialyzed then lyophilized to dried powder.

### Example 8

Synthesis via Active Ester-Dextran of an Extraction Capillary Coated with a Three-Dimensional NTA Extraction Surface

To a solution of polycarboxymethyl dextran (100 mg (dialyzed and freeze-dried, see Example 7) in water (3.0 mL) was added N-hydroxysuccinimide (170 mg) followed by EDAC (290 mg). The reaction continued at RT for 3 hrs. Afterwards there was some grayish precipitate present, which was removed by filtration.

The resulting dextran active ester solution was adjusted to pH ˜8 with 1M NaOH before being pumped through the aminosilane derivatized capillaries of Example 2 at a flow rate of 1 μL/min for 14 hrs (before pumping the dextran solution through the capillary, it was quickly washed with 100 mM NaHCO₃ solution).

The dextran treated capillary was washed with water (0.5 mL; flow rate 100 μL/min) before a solution of NTA in water (200 mM; pH˜8.0; 0.5 mL, as described in Example 4) was pumped through the capillaries. The reaction continued for 4 hrs at RT with the flow rate adjusted to 0.20 mL/h. The capillaries were washed with water (2 mL) before one meter of capillary was removed and charged with Ni2+ as described in Example 6 (single activation).

The capillary was quickly washed with slightly acidic water before being treated with a solution of N-hydroxysuccinimide (170 mg; 1.5 mmol) and EDAC (290 mg; 1.5 mmol) in water (1.5 mL) for 6 hrs with a flow rate of 0.15 mL/h. The capillary was washed with water (0.5 mL; flow rate 0.10 mL/min), then a solution of NTA in water (200 mM; pH˜8.0; 0.5 mL) was introduced into the capillary. The reaction continued for 14 h at RT with the flow rate adjusted 1 μL/min. The capillary was then washed with water (4 mL). The washed capillary was charged with 10 mM NiSO₄ for 20 min as described in Example 6 (double activation).

The effect of single activation vs. double activation on binding capacity was evaluated using the methods of Examples 13 and 18. One meter of the single activated.

### Example 9

Synthesis of HSCH₂CO-NTA

To a solution of mercaptoacetic acid (460 mg; 5.0 mmol) in acetonitrile (14 mL) was added N-hydroxysuccinimide (600 mg; 5.2 mmol) followed with DCC (1.1 mg; 5.5 mmol). The reaction continued for 30 min at RT (it was noted that a substantial amount of ppt formed after a couple minutes of reaction). The insoluble by-product dicyclohexyl urea (DCU) was filtered off and washed with additional acetonitrile (4 mL). The combined colorless product solution was added to a solution of NTA (see Example 4; 175 mM; pH˜8.2; 30 mL; 5.25 mmol; this solution was purged with nitrogen for about 10 min prior to the reaction) and the pH of the reaction mixture adjusted to 8.65 with 1N NaOH. The reaction continued for 3 hrs at RT under nitrogen. The pH of the reaction mixture was readjusted to 2.5 with 6M HCl before filtering. The total volume is 50 mL and assuming 100% yield, the concentration of this solution is 100 mM.

### Example 10

Synthesis of Thiol-Functionalized Capillary

Etched capillaries were prepared as described in Example 1 and were filled with a solution of (MeO)₃Si(CH₂)₃SH (20% in toluene) before being placed in an oven at about 125° C. The reaction continued for 16 hrs with the flow of the silanization solution through the capillary adjusted to 0.15 mL/h. The capillaries were washed with toluene (3000 μL), acetonitrile (2000 μL), water (4 mL), acetonitrile (3000 μL), and dried with nitrogen.

### Example 11

Vinylsulfonedextran Synthesis

Dextran (Fluka, St. Louis, Mo. #31387; MW. 15000-20000; 2 g) was dissolved in water (60 mL) and phosphate buffer (pH 11.5, 400 mM Na₃PO₄, 20 mL) was added to NaBH4 (40 mg), followed by divinylsulfone (5.5 mL; 74 mmol; added all at once). The reaction continued at RT for 27 minutes, then quenched by adjusting the pH to 6 with 6M HCl. The light yellow reaction mixture was dialyzed and lyophilized.

### Example 12

Synthesis via Vinylsulfone Dextran of an Extraction Capillary Coated with a Three-Dimensional NTA Extraction Surface

Vinylsulfone-dextran (Example 11; 200 mg (dialyzed and freezedried)) was dissolved in a solution of 50 mM phosphate buffer (pH=8.5; 3 mL) and DMF (3 mL) was added to clarified the solution. Thiol functionalized capillaries (Example 10; 50 meters×2) were filled with the above solution using 450 psi (it took ˜25 min) and the reaction was allowed to continue for 1 hr at a flow rate through the capillary of 0.5 mL/h.

The dextran treated capillaries were washed with water (2.5 mL each) before reacting with a solution of HSCH₂CO-NTA (Example 9; 100 mM; readjusted to pH 8.5; 3.0 mL per capillary). The reaction continued for 1 hr at RT at a flow rate of 0.4 mL/h. The capillaries were then washed with water (2.5 mL each) and charged with 25 mM NiSO₄ for 20 minutes followed by a solution of 5 mM NiSO₄ in 10% MeOH—H₂O which was used to displace the 25 mM NiSO₄ solution (Example 6). The capillaries are stored at 4° C. filled with 5 mM NiSO₄ in 10% MeOH—H₂O solution.

### Example 13

Procedure for Determining the Capacity of a Ni²⁺-NTA Extraction Capillary via His-GST Protein

A Ni²⁺-NTA capillary of interest is dried with N₂, then loaded with a 20 μL sample plug of a 2500 μg/mL stock solution of His-GST protein (described in U.S. patent application Ser. No. 10/434,713). The sample plug is moved through the capillary two complete cycles with about 2-5 mins of incubation before being expelled from the capillary. The capillary is then washed with water (500 μL; fast flow rate), followed by PBS (20 mM phosphate pH7+140 mM NaCl, 500 μL with about 1 min of incubation) and water (500 μL; fast flow rate). The capillary is then dried with nitrogen for about 1-2 mins.

Next the protein is eluted off the capillary with 200 mM imidazole (15 μL). The imidazole plug is moved through the capillary with two complete cycles with about 2-5 mins of incubation before being expelled from the capillary and collected. 15 μL of water is then added to the collected sample.

The amount of protein in the sample is determined by running sample on an HP1050 HPLC system using a non-porous C-18 column, a gradient of 25% B to 75% B in 5 mins. (solvent A: 0.1% TFA in water and solvent B: 0.1% TFA in acetonitrile) with the detection wavelength of 214 nm, and integrating the protein absorbance peak. A calibration standard is used, which is made by adding 15 μL of a 125 μg/mL protein solution with 15 μL of 200 mM imidazole.

### Example 14

Comparison of Capacities of 3-D and Monolayer Extraction Capillaries

The capacity of a monolayer extraction capillary as described in Example 5 was determined using the method of Example 13. A one meter long section of the capillary was found to bind 1.4 μg of His-GST.

A number of 3-D extraction capillaries as described in Example 5 (of the same length) were tested in the same manner, and were found to typically bind about 10-15 μg of protein. Thus, the 3-D extraction surface results in a substantial improvement in protein binding capacity.

### Example 15

Vinylsulfone Dextran Assay

The purpose of this assay is to determine the amount of vinylsulfone groups in vinylsulfone dextran that are available for further reaction with any nucleophilic thiol group.

This assay is based on the reaction between excess sodium thiosulphate and the available vinyl groups of vinylsulfone dextran. This reaction produces hydroxide ions which can be titrated with hydrochloric acid to determine the level of vinylsulfone substitution for a given amount of vinylsulfone dextran (Journal of Chromatography (1975) 103:49-62).

Experimental Procedure:

1. Accurately weigh out about 100 mg of vinylsulfone dextran.

2. In a 50 mL centrifuge tube, dissolve the vinylsulfone dextran in DMSO (1 mL) and dilute the solution with water (39 mL). The pH of this solution is acidic.

3. Add sodium thiosulphate (800 mg) and shake well.

4. Allow the reaction to proceed for additional 18 hrs on a shaker.

5. Pour the reaction mixture into a 200 mL beaker equipped with a stir bar.

6. Turn on and calibrate the pH meter before placing the probe in the beaker that contains the reaction mixture. The set up is then placed on the stirrer with medium setting.

7. Start titrating with 0.01M hydrochloric acid, with the help of a burette, until the pH of the solution reaches 5.60. Record the total volume of HCl used.

### Example 16

Evaluation of Vinylsulfone Dextran Samples for Concentration of Vinylsulfone Groups and for Protein Binding Capacity

A number of different samples of vinylsulfone dextran were prepared using the method described in Example 11 and assayed using the procedure described in Example 15. The vinylsulfone dextran samples were also used to synthesize 3-D extraction capillaries as described in Example 12 and assayed for His-GST binding capacity using the method of Example 13. The following table provides the mass yield for the vinylsulfonation reactions, the results of vinylsulfone dextran assay for each sample, and the GST capacity for the capillaries corresponding to each sample.

| | Yield in g (all with 2.0 g | μmol of VS/g | μg of GST/m |
|---|---|---|---|
| Sample Name | of starting Dextran) | of VSD | of Cap. |
| VSD042303 | 4.4 | 550 | ˜18 |
| VSD071503 | 2.4 | 534 | 2.4 |
| VSD071603 | 2.7 | 619 | 2.9 |
| VSD072903A | 3.6 | 995 | ˜11 |
| VSD072903B | 3.9 | 1068 | ˜11 |
| VSD072903C | 3.7 | 990 | ˜10 |
| VSD082803A¹ | 2.9 | 495 | 2.7 |
| VSD082803B¹ | 3.0 | 481 | 1.7 |

¹The starting dextran MW. is 6000 instead of 15000-20000 like the rest of the samples.

With the exception of VSD042303, the VS titration results had a direct correlation to the final protein capacity. However, the data was collected over a period of three to four months and there were some variations. These reaction variables include: the integrity of the GST protein as it was shown to degrade over time, the integrity of the thiol-NTA reagent, the amount of available thiol groups on the capillaries, and the experimental variables such as MW of the starting dextran and reaction time.

### Example 17

Determination of Binding Specificity for His-GST in a 3-D Extraction Surface Capillary

20 uL of His-GST protein (1000 ug/mL) was diluted with a solution of 2 mg BSA and 5 mM imidazole in 1 mL of PBS. 500 uL of this mixture was passed through a capillary (3-4 cycles), then washed and eluted with imidazole as described in Example 13. About 7 μg of GST protein was recovered without any detectable BSA.

### Example 18

Determination of the Amount of Ni²⁺ ions Bound to Capillary Surface via 4-(2-pyridylazo) Resorcinol (PAR) Reagent

The objective of this assay is to determine the amount of Ni²⁺ ions bound to capillary surface by chelation to the NTA moieties. Ni²⁺ ions (in aqueous solution) form a stable, colored complex (2:1) with 4-(2-pyridylazo) resorcinol (“PAR”), having λₘₐₓ=495 nm.

The assay is performed on an extraction capillary that has been loaded with Ni²⁺ as described above. A 20 μl slug of 0.01 M HCl is passed through the capillary four times, dissolving the Ni-NTA complex. This effluent is then collected and combined with 20 μl of PAR reagent (4.0×10⁻⁴ M PAR in 3M NH₃, pH=11-12) and incubated for 10 minutes. The sample is analyzed at 495 nm on a FIA flow injection system. Quantification is done via a “one-point” calibration, using 1.0×10⁻⁴ M NiSO₄ in 0.25M HCl as the standard solution.

### Example 19

Determination of Relationship Between Ni²⁺ Capacity and Protein Capacity

The relationship between Ni²⁺ capacity and protein capacity was determined for several different capillaries (see Table), using the procedures of Examples 13 and 18.

Capillary 042203Ni is a Ni-NTA monolayer capillary that was prepared as described in Examples 5 and 6. Capillaries D042303Ni and D042403Ni were prepared using the double activation method of Example 8. Capillary D041003Ni was made by the same procedure as D042303Ni, but the carboxymethyl dextran was used before dialysis and lyophilization. Capillary D042503Ni was produced by the same procedure as D042303Ni, with the exception that the solvent in the reactivation reaction of the attached carboxymethyl dextran was done in acetonitrile instead of water.

As can be seen from Table 1, there is a correlation between nickel chelation and protein binding.

TABLE 1

| Capillary | Ng Chelated | Ug His-GST |
|---|---|---|
| ID No. | Nickel (per M) | Trapped (per M) |
| 042203Ni | 33 | 1.4 |
| D042503Ni | 106 | 5.8 |
| D041003Ni | 137 | 6.3 |
| D042303Ni | 266 | 21 |
| D042403Ni | 320 | 22 |

### Example 20

Preparation of a Strong Acid Cation Exchanger Capillary Channel

A 100 μm ID 50 cm etched fused silica capillary (Polymicro, Inc.) is attached to a syringe pump containing an aqueous 0.1% (v/v) suspension of Biocryl BPA 1000 strong anion exchanger latex (Rohm and Haas, Inc.) and latex is pumped through the capillary at the rate of 100 μL/min for 10 minutes. Then the capillary is flushed with deionized water for 10 minutes, removing the residual anion exchanger. A 0.1% (v/v) aqueous suspension of strong acid cation exchanger, SPR-H (Sarasep, Inc.) is pumped through the capillary at the rate of 100 μL/min for 10 minutes. The capillary is flushed with deionized water for 10 minutes and then put into a refrigerator for storage.

### Example 21

Preparation of a Strong Acid Cation Exchanger Capillary Channel

The process as described in Example 20 is repeated except Biocryl 1050, Rohm and Haas, Inc. is used in place of Biocryl BPA 1000. Biocryl 1050 latex contains both strong base and weak base anion exchanger sites.

### Example 22

Preparation of a Strong Acid Cation Exchanger Capillary Channel

The process as described in Example 20 is repeated except Polybrene® (1,5-dimethyl-1,5-diazaundecamethylene polymethobromide, hexadimethrine bromide) Part Number 10,768-9/Sigma Aldrich, Inc. is used in place of Biocryl BPA 1000 POLYBRENE® is a linear strong base anion exchanger polymer.

### Example 23

Preparation of a Weak Acid Cation Exchanger

The processes as described in Examples 20, 21, and 22 are repeated except a 0.5% (w/v) aqueous suspension of weak acid cation exchanger latex (TWS-3420, Rohm and Haas, Inc.) is used in place of SPR-H.

### Example 24

Synthesis of NTA-Dextran via an Active Ester

To a solution of polycarboxymethyl dextran (150 mg, dialyzed and freeze dried; 0.93 mmol of sugar, see Example 7) in water (5.0 mL) is added N-hydroxysuccinimide (173 mg; 1.5 mmol) followed by EDAC (380 mg; 2.0 mmol). The reaction continues at RT for 60 min before a solution of NTA (see Example 4) in water (175 mM; pH˜8.2; 7.5 mL; 1.2 mmol) is added. The pH of the reaction is then adjusted to about 9 with 0.1M NaOH and the reaction continues for 3 hrs at RT. The pH of the reaction mixture is adjusted back to about 7, and the entire sample is dialyzed and freeze dried.

### Example 25

Synthesis of NTA-Dextran via Vinylsulfone

Vinylsulfone dextran (150 mg, dialyzed and freeze dried, see Example 11) is dissolved in 50 mM phosphate buffer (pH=8.5; 5 mL) and DMF (400 μL). HSCH₂CO-NTA (100 mM; 5 mL, see Example 9) is added to the vinylsulfone dextran solution. The pH of the resulting solution is adjusted to about 8.5 with 1M NaOH. The reaction continues for 1 hr at RT before the pH readjusted to about 6 with 1M HCl and the whole reaction mixture is dialyzed and freeze dried.

### Example 26

Preparation of a NTA Chelator

The processes as described in Examples 23 are repeated except the polymer suspension prepared according to Example 24 or 25 is used in place of SPR-H. A 1% (w/v) aqueous suspension of the polymer is pumped through the coated capillary at a rate of 100 μL/min for 10 mins and then washed with DI water for 10 mins. The capillary is charged with 10 mM NiSO₄ for 10 mins and then washed with DI water for 10 mins.

### Example 27

Synthesis of “Poly-Maleimide” Dextran N-(N′-tert-Butyloxycarbonyl)ethylenemaleimide [M. A. Walker (Tett. Lett., 1994, 35, 665] is treated with trifluoroacetic acid to remove the BOC protecting group. The resulting aminoethylenemaleimide is then acylated with bromoacetylchloride to form N-(N′-bromoacetyl)ethylenemaleimide.

Poly-maleimide dextran is synthesized using an analogous synthetic scheme as was used to synthesize polycarboxymethyl dextran in Example 7, with N-(N′-bromoacetyl)ethylenemaleimide used in place of bromoacetic acid.

The poly-maleimide dextran is then reacted with a protein containing a reactive cysteine group, forming a covalent attachment of the protein to the 3-dimensional dextran matrix (Wang et al. (2003) Bioorganic and Medicinal Chemistry 11:159-6; Toyokuni et al. (2003) Bioconjugate Chem. 14:1253-59; Frisch et al. (1996) Bioconjugate Chem. 7:180-86).

While the invention has been described in connection with specific embodiments thereof, it will be understood that it is capable of further modifications and this application is intended to cover and variations, uses, or adaptations of the invention that follow, in general, the principles of the invention, including such departures from the present disclosure as come within known or customary practice within the art to which the invention pertains and as may be applied to the essential features hereinbefore set forth. Moreover, the fact that certain aspects of the invention are pointed out as preferred embodiments is not intended to in any way limit the invention to such preferred embodiments.

What is claimed is:

1. An extraction capillary channel, wherein a substantial portion of the channel is coated with a 3-dimensional solid phase extraction surface that binds an analyte.

2. The extraction capillary channel of claim 1, wherein the analyte binding capacity of the 3-dimensional solid phase extraction surface is greater than could be achieved by a corresponding 2-dimensional solid phase extraction surface.

3. The extraction capillary channel of claim 1, wherein the solid-phase extraction surface comprises a polymer.

4. The extraction capillary of claim 3, wherein the polymer is covalently attached to the capillary channel.

5. The extraction capillary of claim 3, wherein the polymer is non-covalently attached to the capillary channel.

6. The extraction capillary of claim 5, wherein the polymer is attached to the capillary channel by electrostatic interaction.

7. The extraction capillary of claim 6, wherein the polymer is attached to the capillary channel by electrostatic interaction to a second polymer, wherein the second polymer is attached to the capillary channel.

8. The extraction capillary of claim 7, wherein the second polymer is attached to the capillary channel by electrostatic interaction.

9. The extraction capillary of claim 6, wherein the polymer is a bead.

10. The extraction capillary of claim 9, wherein the polymer is a latex bead.

11. The extraction capillary channel of claim 3, wherein the polymer is a polysaccharide.

12. The extraction capillary channel of claim 3, wherein the polymer is dextran.

13. The extraction capillary channel of claim 1, wherein an extraction agent is attached to the solid-phase extraction surface.

14. The extraction capillary channel of claim 1, wherein the extraction agent is an immobilized metal, a protein, or an antibody.

15. The extraction capillary channel of claim 1, wherein the analyte is a biomolecule

16. The extraction capillary channel of claim 15, wherein the biomolecule is a protein.

17. The extraction capillary channel of claim 1, wherein the capillary channel is fused silica capillary tubing.

18. The extraction capillary channel of claim 3, wherein an extraction agent is covalently attached to the polymer.

19. The extraction capillary of claim 18, wherein the extraction agent is Ni-NTA, Protein A or Protein G.

20. The extraction capillary of claim 3, wherein the 3-dimensional solid phase extraction surface can be penetrated by a biomolecule analyte having a molecular weight of 2000.

21. A method for preparing an extraction capillary channel having a 3-dimensional extraction surface, comprising the steps of:
a) providing a capillary channel bearing a first attachment group; and
b) attaching an extraction polymer to said capillary channel by an interaction between said first attachment group and a second attachment group on said extraction polymer, wherein said extraction polymer bears an affinity group having an affinity for an analyte.

22. The method of claim 21, wherein said extraction polymer is attached to said capillary channel by formation of a covalent bond between said first and second attachment groups.

23. The method of claim 22, wherein said covalent bond is an amide bond, an isourea bond or a thioether bond.

24. The method of claim 21, wherein said extraction polymer is attached to said capillary channel by an electrostatic interaction between said first and second attachment groups.

25. The method of claim 21, wherein said extraction polymer is dextran.

26. The method of claim 21, wherein said extraction polymer is a latex bead.

27. A method for molecular open tubular solid phase extraction, the method comprising the steps of:
a) adsorbing analyte molecules in a sample solution to the extraction surface of a fused silica extraction capillary tubing of claim 1, the capillary tubing having a total capillary volume; and
b) desorbing a substantial portion of the analyte molecules from the extraction surface with a desorbent liquid passed through the capillary channel.

28. The method of claim 27, wherein the analyte molecules is desorbed with a Tube Enrichment Factor of at least 1.

29. The method of claim 27, wherein the direction of passage of the desorption solution through the column reversed during the desorption step.

30. The method of claim 27, wherein a wash solution is passed through the capillary channel between steps (a) and (b).

31. The method of claim 27, wherein the wash solution is any liquid present in the capillary channel is substantially displaced from the capillary channel by a gas before step (b).

32. The method of claim 31, wherein the direction of passage of the gas through the column is reversed during displacement of the liquid.

33. The method of claim 27, wherein the extraction surface has an affinity binding agent bound thereto, and the affinity binding agents is:
a) a chelated metal having a binding affinity for a biomolecule analyte;
b) a protein having a binding affinity for a protein analyte;
c) an organic molecule or group having a binding affinity for a protein analyte;
d) a sugar having a binding affinity for a protein analyte;
e) a nucleic acid having a binding affinity for a protein analyte;
f) a nucleic acid or a sequence of nucleic acids having a binding affinity for a nucleic acid analyte; or
g) a small molecule binding agent having a binding affinity for a small molecule analyte.

34. The method of claim 27, wherein the analyte concentration is increased at least 100 times.

35. The method of claim 27, wherein the analyte molecules are desorbed with a Tube Enrichment Factor from within a range from 1 to 400.
